(19) United States
(12) Patent Application Publication
(10) Pub. No.: US 2016/0349229 A1
(43) Pub. Date: Dec. 1, 2016

(54) Hand-Held Pesticide Analysis Zeroize Button the Whole Plant with Non-Enzymatic Determination of Pesticides for Disposable Non-Enzymatic Multiple Analysis Set Kit

(71) Applicant: Betül Yasemin Akar, Izmir (TR)

(72) Inventor: Betül Yasemin Akar, Izmir (TR)

(21) Appl. No.: 15/233,977

(22) Filed: Aug. 11, 2016

Related U.S. Application Data
(63) Continuation of application No. PCT/TR2015/000141, filed on Apr. 3, 2015.

(51) Int. Cl. G01N 33/00; G01N 27/327; G01N 27/403

(52) U.S. Cl. CPC G01N 33/0098; G01N 27/403; G01N 27/327; G01N 2430/10

(57) ABSTRACT

Hand-held pesticide analysis zeroize button the whole plant with non-enzymatic determination of pesticides for disposable non-enzymatic multiple analysis set kit. This invention about hand-held pesticide analysis in non-enzymatic determination of pesticides in all plant parts for single-use non-enzymatic multiple analysis set kit and kit to be used in non-enzymatic analysis method of multiple pesticides.

### RELATED APPLICATIONS

This application is a continuation application of International Application No. PCT/TR2015/000141, filed Apr. 3, 2015.

The above application and all patents, patent applications, articles, books, specifications, other publications, documents, and things referenced herein are hereby incorporated herein in their entirety for all purposes. To the extent of any inconsistency or conflict in the definition or use of a term between any of the incorporated publications, documents, or things and the text of the present document, the definition or use of the term in the present document shall prevail.

### BACKGROUND OF THE INVENTION

Field of Invention

The present invention relates to hand-held pesticide analysis, particularly to hand-held pesticide analysis in non-enzymatic determination of pesticides in all plant parts for single-use non-enzymatic multiple analysis set kit and kit to be used in non-enzymatic analysis method of multiple pesticides.

Related Art

Determination of pesticides in agricultural products in the analysis are still maintained in the laboratory. Determination of pesticides in agricultural products in the analysis are still maintained in the laboratory. Brought to the lab for analysis standard samples if the sampling method, the filter being here decortigated and centrifugal process are being treated with the chemical solution being migrated from, purify it, and analysis. Besides preliminary preparation operations are performed analysis tools and materials are different from each other and not in a set has been available in the laboratory, the individually. Peak season due to the increased workload, additional staff requirement for these operations. A specimen at sometimes different groups of pesticide analysis is done, the cost of the transaction process and significantly increases. Analysis of pesticide in the field for many companies by organophosphates and urethane containing acetylcholine, butyrylcholine for use in the determination of pesticides and so developed a single-use enzymatic kits containing enzyme inhibitor. In accordance with the instructions for use with this kit prepared samples containing urethane compounds organophosphate or contained in the kit are appointed with the biochemical field color change.

The aforementioned kit comes with compounds containing the pesticide active ingredients cannot be identified individually, and the amount of pesticide metabolites cannot be transferred. In this analysis, based on the principle of yes/no pesticide detected is far above legal limits MRL values. It also contains compounds in agricultural production except urethane organophosphate and organochlor, halogen and oxygen containing compounds that contain sulfur, copper, tin, dinitrofen and derivatives, dinitroa analysis, chlorinated aliphatic acid, my triazol, belongs to groups such as uracil compounds triazin is the pesticides are still used. This enzymatic methods all of the pesticides it is not possible to determine. Surface coated with pesticides and pesticide metabolites of the electrode legal MRL values and to identify further down levels, differentiate and determine the amount and variety of ways to increase the precision of the electrode is possible.

One preferred embodiment of the invention was taken for analysis from the field conditions plant sample, non-enzymatic multiple pesticide analysis method in accordance with the multiple analysis set kit is being torn apart by means of the apparatus, except the pesticide active ingredient groups sought the example is a filter unwanted molecules, solids have been collapsing, determination of pesticide active ingredient in different cells within the kit being distributed, within the cell analysis will be done with the active substance be subject to special treatment, chemical buffer solutions, polymerised and over coated electrode with electro-chemical reaction is being maintained and subject to set outside the electrode terminals of handheld pesticide by reading these reactions on connecting to data. Non-enzymatic method is used to set multiple pesticide analysis is specialised kit.

### SUMMARY OF THE INVENTION

One objective of a preferred embodiment of the invention, before the harvest of the plant pesticide and pesticide metabolites in all parts of the non-enzymatic detection and determination can be made, leaves, fruits from the harvested plant yet, a single set of samples, such as sap, roots for the apparatus to be brought together with fragmentation, soar, filtering, sampling is automatically activated in cells where the different pesticides distributed cell to be treated with a chemical inside the buffer solutions, electrode surface coated and polymerised with electro- and chemical reaction is formed by connecting to handheld pesticide analysis device will allow you to read and the data to be converted into disposable non-enzymatic analysis set set is to perform.

Another objective of a preferred embodiment of the invention also hand-held pesticide analysis device plant crops, such as stalks and leaves taken from the parts of the kit cover bezel from the private placement to divide, plant particles here and be homogenized with knives, steel knives with a stop at the entrance to the analysis in the split opened cells through vacuum filter, analyze, and polymerized in the cells treated with the pesticide active ingredient have different buffer solutions, environment and each pesticide active ingredient group with specialized gas electrochemical properties of protection located in cell surface analysis of each analysis requested active according to the article and to enter a chemical reaction in the electrode, consisting of polymerized chemical reaction can be seen in the data insertion device of the kit becomes readable and with hand-held non-enzymatic multiple pesticide analysis device is to perform multiple pesticide analysis method.

It is another object of a preferred embodiment of the invention to provide a simple, efficient, durable, and cost effective hand-held pesticide analysis device.

### BRIEF DESCRIPTION OF THE DRAWINGS

FIG. 1 is a schematic view of one preferred embodiment of the hand-held pesticide analysis device.

### DETAILED DESCRIPTION OF THE INVENTION

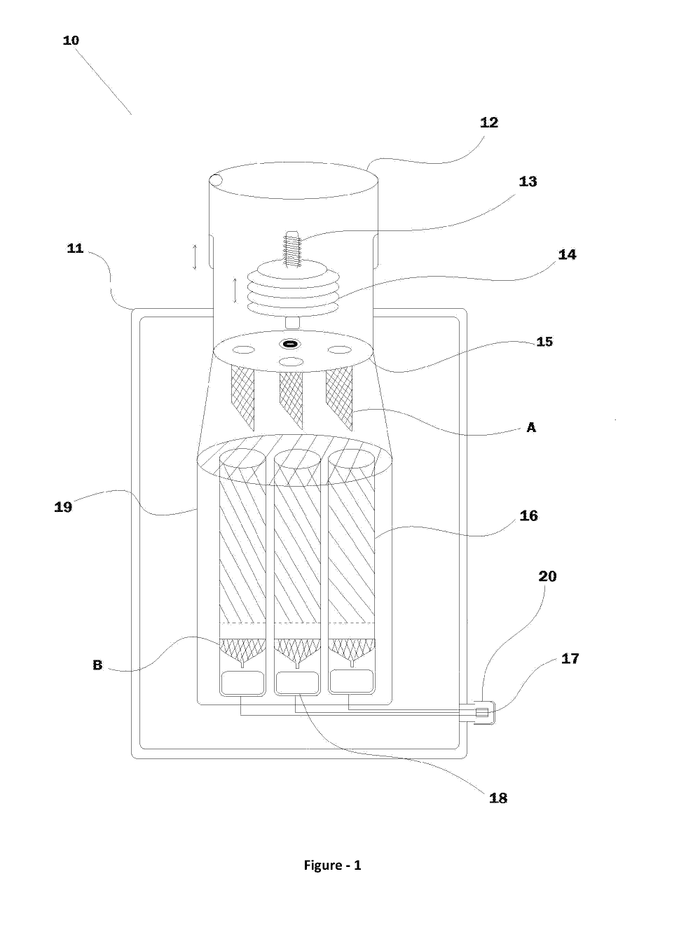

As shown in FIG. 1, a handheld pesticide analysis device 10 in non-enzymatic determination of pesticides in plant parts for single-use non-enzymatic and non-enzymatic multiple analysis kit multi-analysis method, durable and made of transparent material and part of a torso that can color on demand; the underlying mechanism is automatic on/off and the front and the sample is placed on the upper lid 12 and lower lid 15 from a reservoir; the cover of the tank and opened after the mechanism and rotating stainless steel blades non-enzymatic multiple pesticide analysis method and preliminary mixing, for example, in a manner appropriate to process the handling pane; and blended sample of polymerised and hole diameter may vary automatically in each vacuum filter A through a custom bumper with chemicals and pesticide group analysis for surface coated and polymerised electrodes 18 and some, or all, of the different groups of pesticides, including private gas will be transferred from the Division of the cell 16; out of the output of each cell 16 are fly and fly the electrode 18 has been provided for transparent and durable material consists of a cover part.

The plant sample is loaded in the chamber of the upper and lower covers are opened as interconnected, non-enzymatic multi analysis methods, according to the analysis of the samples will be determined by the amount of anterior process, and will be the standard.

Processing compartment located inside the spring and rotating stainless steel blades non-enzymatic multi analysis methods, according to the plant's stem, branches, leaves and fruit are examples of homogeneous parts and in a format that can be imported will be confused. These knives 14 are spring pressure and rotation can be set in advance. Opening the lid 20, allowing the transition from processing portion of the vessel premises blades let loose the spring mechanism 13 that compresses and preset number and rapidly fragmenting the sample by working on the blades.

Blades rotation stops processing tank is located under the cover opening of launch and non-enzymatic mechanism of multi-analysis method, according to the analysis to be done under the cover of plant sample can vary from polymerised and vacuum the filter A hole diameter cells 16. Filters A-B to analyze the environment hinders the passage of unwanted ions into the cells 16.

Analysis and process of a filter A-B that filters B are polymerized to increase the sensitivity. Cells 16 in non-enzymatic multi analysis methods, according to the analysis to be done pesticide active ingredients to look for in a custom buffer solutions and over to the Group of active ingredients will be coated with material that react with electro-chemical electrode 18. When cells 16 that can accommodate specialized pesticide active ingredient group gas, too.

Electro-chemical analysis of the body of the electrode 18 connection end 17 is part of the set is invulnerability is emitted out of order a lid 20 for protection.

Search multiple electrodes 18 connection analysis kit multiplexer to connect to handheld pesticide volume devices and non-enzymatic reaction consisting of multiple analysis methods are read on the device, according to the data.

Multiple analysis kit bowl 19, chopper, mixer blades and active gas analysis is as a set that contains the cells 16 of non-enzymatic multiple analysis not only according to the method of the plant sap, leaves, stalks, fruit, as well as the intended analysis of a combination of components. The set is non-enzymatic analysis of acetylcholine, butyrylcholine etc. without the use of enzymes and biological sensors, electrochemical methods are intended to be made.

The set is a single-use, non-enzymatic multi analysis methods, according to a sterile environment at the time of the analysis, the infestation and resulting from the previous analysis of microbes reproduction without question of intended in a reliable manner.

The set is made of durable and transparent materials of the part of the body that is intended to be visible to the process were made.

Set the color of the part of the body, will be prepared according to the different groups of active ingredients of pesticides analysis kits are and ease of use.

With a covered bowl 19 on the set; non-enzymatic multi analysis methods, according to the analysis of the plant to be held within the designated standards for analysis is intended to be placed on the set.

Bowl 19 with cover, non-enzymatic mechanism under multi-analysis method according to the hopper the hopper of the konan plant with the top cover off by working of the mechanism of specimens and the shredding and mixing of switching blades will run the spring mechanism 13 is intended to the introduction.

Then the spring loaded mechanism and stainless steel blade, being part of the processing, non-enzymatic multiple analysis of the plant according to the method of fragmentation and homogenized in a format intended for mixing. With stainless steel knives 14, knives 14 is intended to be sturdy and durable.

These knives 14 are spring loaded, being, according to the method of multiple analysis cover non-enzymatic mechanism, depending on the case, the speed of rotation and the adjustable is intended to be.

Processing under the knives 14 of the stop is activated with the mechanism cover, non-enzymatic multiple analysis according to the method of fragmenting and dividing the fifteen samples involved in the analysis of the cells 16 at the entrance to the polymerized intended to transition filters B.

Is that filters A-B are polymerized and vacuum according to the method of multiple, non-enzymatic analysis of samples during the transition to the underlying cell 16 vacuum force keeping unwanted molecules for analysis by filtering and filter B is intended to increase the sensitivity of the process.

In the pane at the bottom of the set, in the form of the analysis cell 16 compartments, being, according to multiple non-enzymatic analysis method in a single analysis of different groups of active ingredients of pesticides is intended to be made.

In cells 16 with buffer solutions, according to the method of analysis of the multiple non-enzymatic analysis will be done to the electrode 18 reaction of active ingredients intended to increase can give. Within the cell 16 is non-enzymatic chemical electro-electrodes 18 according to the method of pesticide active ingredient analysis of multiple electrodes 18 is intended to react.

Electrode 18 coating material using non-enzymatic analysis will be made according to the method of multiple analysis of pesticide active ingredient is intended to the detectability.

The electrode 18 being polymerized non-enzymatic reaction according to the method of multiple analysis requested the withdrawal of unwanted ions from the atmosphere depending upon electrode 18 be pushed down so that it is intended to increase the sensitivity of the electro-chemical reaction.

Some or all the cells 16 in the active substance when needed custom gas according to the method of multiple non-enzymatic analysis of assets intended to protect properties of reacting.

Set of electrodes 18 in cells 16 outside of the body of the endings with the analysis kit, a multiplexer units of handheld device and the connection between cells 16 by reading data is intended to be brought into the reaction.

At the bottom of the hull is made of transparent and durable material with a lid 20, the open ends of the electrodes 18 and is intended to protect from external factors.

The plant's fruit, stalks and leaves as well as the parts of the kit received the bezel from the cover placing special compartments according to the method of multiple non-enzymatic analysis intended to start the analysis.

Plant particles and homogenized with steel blades here, according to multiple non-enzymatic analysis methods at the entrance to the next stage of analysis cells 16 can easily pass through filters A and vacuum and polymerized in the cell 16 buffer solutions needs to be treated equally with the intended.

From here the analysis drop-down pane with knife stop cells from the entrance to the polymerized and passing of vacuum filter A according to the method of analysis of the multiple non-enzymatic analysis of plant made on-site at a molecular level, keeping unwanted impurities contained ions and intended as healthy in the analysis.

Analysis of different pesticide active substances in cells 16 treated with buffer solutions should be according to the method of analysis of the multiple non-enzymatic analysis will be made of the active ingredients of pesticides is intended to react with buffer solutions.

Environment and each pesticide active ingredients with specialized gas to the group, according to the analysis, multiple analysis of non-enzymatic methods sample within the pesticide active ingredient intended for the protection of the electrochemical properties of ions.

Located in cell 16 surface analysis of each analysis requested active according to the article and the introduction of chemical reaction in the electrode 18, non-polymerized enzymatic analysis of multiple samples according to the method of analysis to be made in the amount of pesticide active ingredient of presence and intended to be identified.

This non-enzymatic chemical reaction that occurs as a result of multiple analysis method, the kit can be viewed on the device connecting a intended to become.

The invention to industry application format

Who serve the purposes mentioned above, hand-held pesticide analysis in non-enzymatic determination of pesticides in all plant parts for single-use non-enzymatic and non-enzymatic multiple analysis kit multi-analysis method in the industry can be produced with any or several branches, can be applied to industry

### LIST OF REFERENCE NUMERALS

10 analysis device
11 body part
12 upper lid
13 spring mechanism
14 knife
15 lower lid
16 cell
17 connection end
18 electrode
19 bowl
20 lid
A filter
B filter

What is claimed is:

1. A handheld pesticide analysis device (10) in non-enzymatic determination of pesticides in plant parts for single-use non-enzymatic multiple analysis set kit and this set to use non-enzymatic multiple pesticide analysis method and property; durable and made of transparent material and optional colored body part (11); the underlying mechanism is automatic on/off and the front and the sample is placed on the upper and lower clamshell in a bowl (19); working with the spring cover the hopper mechanism and rotating stainless steel blades; for example, mixing process of occurrence of processing fragmented front pane; the occurrence of the blended sample as fragmented homogenized polymerised and hole diameter change vacuum filters (A-B); the filter (B) automatically in each pesticide group through specialized buffer chemicals; to analyze the cells (16) in the surface of coated electrodes (18) and polymerised; some or all of the cells (16) in the active item-specific gas; different pesticide active ingredient will be assigned to groups of cells (16) that divide; out of the output of each cell (16), electrode (18) tips are provided; electrode (18) tip is made of transparent and durable materials for safekeeping of the cover part.

2. A handheld pesticide analysis device (10) according to claim 1, hand-held pesticide analysis in non-enzymatic determination of pesticides in all plant parts for single-use non-enzymatic multiple analysis set kit is to be used in this set and non-enzymatic multiple pesticide analysis method and property; durable and made of a transparent material and contain colored body part, on demand.

3. A handheld pesticide analysis device (10) according to claim 2, hand-held pesticide analysis in non-enzymatic determination of pesticides in all plant parts for single-use non-enzymatic multiple analysis set kit is to be used in this set and non-enzymatic multiple pesticide analysis method and property; the underlying mechanism is automatic on/off and the front and the sample is placed on the upper (12) and lower lid (15) is a reservoir.

4. A handheld pesticide analysis device (10) according to claim 3, hand-held pesticide analysis in non-enzymatic determination of pesticides in all plant parts for single-use non-enzymatic multiple analysis set kit is to be used in this set and non-enzymatic multiple pesticide analysis method and property; working with the spring cover the hopper mechanism and rotating stainless steel blades.

5. A handheld pesticide analysis device (10) according to claim 4 hand-held pesticide analysis in non-enzymatic determination of pesticides in all plant parts for single-use non-enzymatic multiple analysis set kit is to be used in this set and non-enzymatic multiple pesticide analysis method and property; for example, mixing process of occurrence of processing fragmented front pane.

6. A handheld pesticide analysis device (10) according to claim 5, hand-held pesticide analysis in non-enzymatic determination of pesticides in all plant parts for single-use non-enzymatic multiple analysis set kit is to be used in this set and non-enzymatic multiple pesticide analysis method and property; the occurrence of the blended sample as fragmented homogenized polymerised and hole diameter change vacuum filters (A-B).

7. A handheld pesticide analysis device (10) according to claim 6, handheld pesticide analysis in non-enzymatic determination of pesticides in all plant parts for single-use non-enzymatic multiple analysis set kit is to be used in this set and non-enzymatic multiple pesticide analysis method and property; through the filter (B) automatically in each group of pesticides contain chemicals that are in a custom bumper.

8. A handheld pesticide analysis device (10) according to claim 7, hand-held pesticide analysis in non-enzymatic determination of pesticides in all plant parts for single-use non-enzymatic multiple analysis set kit is to be used in this set and non-enzymatic multiple pesticide analysis method and property; to analyze the cells (16) in the surface of the coated and polymerised electrodes (18).

9. A handheld pesticide analysis device (10) according to claim 8, hand-held pesticide analysis in non-enzymatic determination of pesticides in all plant for disposable non enzymatic multiple analysis set and this set to use non-enzymatic multiple pesticide analysis method and property; some or all of the cells (16) in the active item-specific gas.

10. A handheld pesticide analysis device (10) according to claim 9, the hand-held pesticide analysis device (10) non-enzymatic determination of pesticides in all plant parts for single-use non-enzymatic multiple analysis set kit is to be used in this set and non-enzymatic multiple pesticide analysis method and property; different pesticide active ingredient will be assigned to groups of cells that are dividing.

11. A handheld pesticide analysis device (10) according to claim 10, hand-held pesticide analysis in non-enzymatic determination of pesticides in all plant parts for single-use non-enzymatic multiple analysis set kit is to be used in this set and non-enzymatic multiple pesticide analysis method and property; out of the output of each cell (16) is the tips of the electrodes (18) has been provided.

12. A handheld pesticide analysis device (10) according to claim 11, hand-held pesticide analysis in non-enzymatic determination of pesticides in all plant parts for single-use non-enzymatic multiple analysis set kit is to be used in this set and non-enzymatic multiple pesticide analysis method and property; electrode (18) tip is made of transparent and durable materials for safekeeping of the cover part.

13. A handheld pesticide analysis device (10) according to claim 12, hand-held pesticide analysis in non-enzymatic determination of pesticides in all plant parts for single-use non-enzymatic multiple analysis set kit is to be used in this set and non-enzymatic method that is multiple pesticide analysis feature: plant crops, such as stalks and leaves on the cover of the parts kit received the bezel from the private placement to divide, plant particles here and be homogenized with knives (14), steel knives (14) with a stop at the entrance to the analysis in the split opened cells (16) pass through filters (A) and vacuum polymerized the analysis of different pesticide active substances in the cells (16) to be treated, setting, buffer solutions, and each pesticide active ingredient group with specialized gas electrochemical property located in each analysis the protection of cell (16) surface covered by item and requested the analysis of active polymerizing electrode (18) into the chemical reaction in the chemical reaction that occurs can be read on connecting to the device, kit and method of data become visible.
